(19) United States
(12) Patent Application Publication
(10) Pub. No.: US 2024/0253414 A1
(43) Pub. Date: Aug. 1, 2024

(54) MITIGATION OF KINETOSIS IN A MOVING VEHICLE

(71) Applicant: ClearMotion, Inc., Billerica, MA (US)

(72) Inventor: Marco Giovanardi, Melrose, MA (US)

(73) Assignee: ClearMotion, Inc., Billerica, MA (US)

(21) Appl. No.: 18/560,725

(22) PCT Filed: May 16, 2022
(86) PCT No.: PCT/US2022/029399

§ 371 Date: Nov. 14, 2023

Related U.S. Application Data
(60) Provisional application No. 63/189,322, filed on May 17, 2021.

(51) Int. Cl. B60G 17/0195; B60G 17/016; B60N 2/02; G06F 3/01

(52) U.S. Cl. CPC B60G 17/0195; B60G 17/016; B60N 2/0278; G06F 3/012; G06F 3/013; B60G 2400/0511; B60G 2400/0512

(57) ABSTRACT

Various systems and methods are disclosed for anticipating the occurrence and/or mitigating the severity and/or duration of kinetosis experienced by one or more occupants of a vehicle. This is especially while the vehicle is travelling over a road surface and one or more occupants are performing tasks that require visual focus. Also, disclosed are systems and methods for predicting or otherwise determining aspects of head motion and characteristics of the eye motion of an occupant of a vehicle and of using that information to mitigate kinetosis by controlling an aspect of vehicle motion.

### RELATED APPLICATIONS

This application claims the benefit of priority under 35 U.S.C. § 119(e) of U.S. Provisional Application Ser. No. 63/189,322, filed May 17, 2021, the disclosure of which is incorporated herein by reference in its entirety.

### BACKGROUND

Motion sickness, or kinetosis, is a phenomenon experienced by the occupants of many vehicles travelling by sea, land, rail, or in space. It is well known that the driver of, for example, a car is much less likely to suffer from kinetosis than occupants not controlling the vehicle. Traditionally, motion sickness was believed to occur when the brain received conflicting information from multiple senses.

The causes of kinetosis are not understood completely, but many contributing factors have been established in research. A mismatch between inner ear organs and other sensory input such as for example visual or tactile input is one important factor, such as is a mismatch between expected and perceived motion. Other factors known in the industry include low frequency motions, rotations of the head, and combined motions including rotations and displacements.

### SUMMARY

According to aspects of the disclosure, there is provided a method for operating a vehicle that includes: transporting an occupant of the vehicle over a road surface, while the occupant is seated on a seat in the vehicle; measuring a road-surface-induced disturbance of a portion of the vehicle, with a first sensor; based on one or more measurements with the sensor, with a first model that relates the motion of the occupant's head to the road-surface-induced disturbance, determining an aspect of a motion of the occupant's head (e.g., pitch motion of the occupant's head, roll motion of the occupant's head, or heave motion of the occupant's head); based on the determined aspect, controlling a motion of the seat with a microprocessor-based controller; and controlling the aspect of the determined motion with the motion of the seat. In some embodiments, control of the aspect of the occupant's head may include controlling an amplitude of the motion. In some embodiments, the amplitude of the motion may be controlled in a frequency range between 1 Hz and 9 Hz, or alternatively in a frequency range between 3 Hz and 8 Hz. In some embodiments, a seat actuator may be interposed between the seat and the vehicle floor, and may be used to control the motion of the seat. Alternatively or additionally, in some embodiments, a vehicle active suspension actuator may be interposed between an unsprung mass of the vehicle and a sprung mass of the vehicle, and may be used to control the motion of the seat. In some embodiments, the first model may include a first transfer function that relates a movement of the occupant's head to a movement of the seat. In some embodiments, the first model may also include a second transfer function that relates the movement of the seat to a movement of the sprung mass. In some embodiments, the first model may also include a third transfer that relates the movement of the sprung mass to a movement of the unsprung mass. In some embodiments, the microprocessor-based controller may be a feedback controller that receives information from the first model.

According to aspects of the disclosure, there is provided a method for operating a vehicle that includes: transporting an occupant of the vehicle over a road surface, wherein the occupant is seated on a seat in the vehicle and performing a focal visual task; measuring a road-surface-induced disturbance of a portion of the vehicle, with a first sensor; based on the measurements, determining, with a first model, a value of at least one parameter (e.g., fixation rate, a forward saccade ratio, a saccade amplitude, a backward saccade amplitude, vision error, or VOR suppression) associated with a motion of an eye of the occupant during performance of the focal visual task, where the first model relates the motion of the occupant's eye to the disturbance; based on that determination, controlling a motion of the seat with a microprocessor-based controller; and controlling the value of the parameter with the motion of the seat. In some embodiments a seat actuator may be interposed between the seat and the vehicle floor and may be used to control the motion of the seat. Alternatively or additionally, a vehicle active suspension actuator may be interposed between an unsprung mass of the vehicle and a sprung mass of the vehicle and used to control the motion of the seat. In some embodiments, the first model may include a first transfer function that relates a movement of the occupant's eye to a motion of the occupant's head. In some embodiments, first model may also include a second transfer function that relates a movement of the occupant's head to a movement of the seat. In some embodiments the first model may also include a third transfer function that relates the movement of the seat to a movement of the sprung mass. In some embodiments, the first model may also include a fourth transfer that relates the movement of the sprung mass to a movement of the unsprung mass. In some embodiments, microprocessor-based controller may include a feedback controller that receives information from the first model.

According to aspects of the disclosure, there is provided a method for operating a semi-autonomous vehicle including: determining that, in a first mode of operation of the vehicle, an occupant in the semi-autonomous vehicle is driving the vehicle; operating a suspension actuator, in the vehicle (e.g., a seat suspension system actuator or a vehicle active suspension system actuator) with a microprocessor-based controller, in the first mode of operation of the vehicle, where a control parameter of the controller is set at a first value; determining that, in a second mode of operation of the vehicle, the occupant is not driving the vehicle; and operating the suspension actuator, in the second mode of operation of the vehicle, with the microprocessor-based controller, where the control parameter of the controller is set at a second value. In some embodiments, the first value is selected based on a vehicle metric selected from the group consisting of handling, vehicle component wear, and efficiency. In some embodiments, the second value is selected based on a vehicle a comfort level of the occupant. In some embodiments, the method may further include determining that the occupant is performing a focal visual task; and during the performance of the focal visual task, operating the suspension actuator with a microprocessor-based controller, where the control parameter of the controller is set at a third value. In some embodiments, the third value is selected to, for example, reduce a motion of the occupant's head, a vision error of the occupant, or VOR suppression by the occupant.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 shows a schematic representation of a system model that relates road input to performance metrics for a road vehicle.

FIG. 2 shows an example of a road input accounting for typical road contour and for wheelbase filtering.

FIG. 3 shows an example of a modeled response of a road vehicle from road input to vehicle body acceleration, in a single degree of freedom.

FIG. 4 shows an example of a model of a seat, torso, and neck in a single degree of freedom for an occupant sitting in a typical road vehicle seat.

FIG. 5 shows an example of a full model showing the relationship between typical road acceleration and head acceleration of an occupant.

FIG. 6 shows an example of data measured to estimate the fixation mechanism in a person's eye.

FIG. 7 shows a schematic representation of an occupant sitting on a platform and focusing their gaze on a handheld target.

FIG. 8 shows a possible representation of the eye motion mechanisms in play when a subject focuses their gaze on a target while their head and the target are in motion.

FIG. 9 shows an example of a predicted transfer function from typical road input to the effort used in suppressing the VOR while focusing on a target.

FIG. 10 shows the same predicted transfer function with a comparison between two different control strategies in the suspension control system of the modelled vehicle.

FIG. 11 shows an example of a predicted transfer function from typical road input to the vision error when the occupant is focusing on a target, for two different control strategies used in the suspension system.

FIG. 12 shows an example of a predicted transfer function from typical road input to the acceleration of the head when the occupant is focusing on a target, for two different control strategies used in the suspension system.

FIG. 13 shows an example of a predicted transfer function from typical road input to the acceleration of the head as a combination of both heave and roll motions when the occupant is focusing on a target, for two different control strategies used in the suspension system.

FIG. 14 shows a schematic layout of a possible strategy for increasing the predictability of the behavior of the platform while at the same time managing the travel excursions of the platform's suspension system.

### DETAILED DESCRIPTION

The Inventors have recognized that under certain circumstances, a contributing factor or the predominant contributing factor, to kinetosis or motion sickness, may be the performance of focal visual tasks in a moving vehicle. Performing tasks that require visual focus, in a moving vehicle, is more likely to induce kinetosis than relaxing or sleeping. Focal visual tasks may involve, without limitation, reading, writing, drawing, watching a screen or an object, reading email, messaging, and/or watching video.

Being able to perform visual tasks efficiently may be important to a typical vehicle occupant, especially as more and more vehicle occupants are not vehicle operators. This is frequently the case in commercial air transport and marine transport, and rail transport, and with increasing frequency in the case of ride-sharing or traveling in autonomous or semi-autonomous road vehicles. There is, therefore, a growing need for methods and systems for delaying or preventing the onset of kinetosis and, if appropriate, of mitigating its severity.

Vehicles (e.g., boats, trains, and on-road and off-road vehicles (e.g., cars, SUVs, vans, trucks, buses)) may include systems such as propulsion, braking, steering and/or suspension systems (e.g., vehicle fully active suspension, vehicle semi-active suspension, seat suspension, seat active suspension) that are configured to control or alter the motion of one or more portions of a vehicle. The Inventors have recognized that such systems may be used to alter the motion experienced by vehicle occupants to delay or prevent the onset and/or mitigate the severity or duration of kinetosis.

In some embodiments, kinetosis may be mitigated by reducing or effectively eliminating certain types of motions for example in certain frequency ranges, adding specific types of motions, and/or altering specific types of motions. A moving vehicular platform may be, for example, the body of a moving road vehicle, a train, a ship, or may be a payload platform on a moving vehicle such as for example a transport bed on a sleeper train, bus, or ambulance. Alternatively, it may also be a vehicle in an amusement park, or in a mine or other production or extraction facility such as a warehouse, an assembly plant, or other.

A platform, that is a part of a moving vehicle, may be fitted with a controller system (including one or more microprocessor-based controllers) configured mitigate or otherwise alter the motion of the platform by using, for example, actuators in various systems such as propulsion, braking, steering and/or suspension systems (e.g., vehicle fully active suspension, vehicle semi-active suspension, seat suspension, active seat suspension). While it is generally understood that less motion may be desirable for avoiding, delaying, or mitigating kinetosis, certain types of motions are an inherent part of traveling in a moving vehicle. Therefore, certain tradeoffs may sometimes need to be relied upon reduce the occurrence of kinetosis as a result of performing a focal visual task in a moving vehicle. The current disclosure describes methods by which the benefits of such tradeoffs may be investigated, identified and/or implemented.

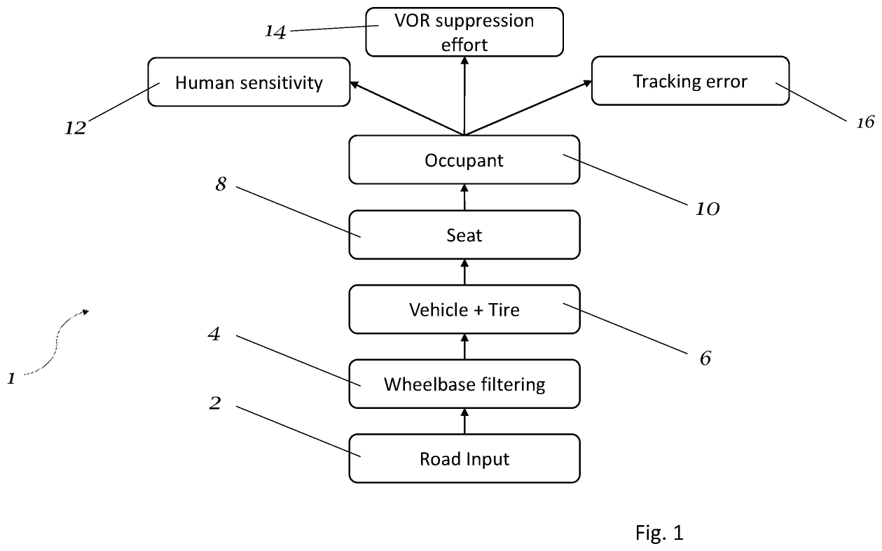

Inventors have recognized that, when a vehicle occupant is performing a focal visual task while traveling along a road surface in a vehicle, interactions between the vehicle and the road surface may induce certain motions in the occupant's body, including the occupant's head and/or eye(s). Furthermore, the Inventors have recognized that some of the head and eye motion may hasten the onset, increase the likelihood and/or increase the intensity or duration of kinetosis. FIG. 1 shows a block diagram of an exemplary transmission chain for a road vehicle. The Inventors have recognized that a model (which may include mathematical, empirical, or combination of mathematical and empirical sub-models) of such a transmission chain may be constructed for any type of vehicle. The descriptions here are for illustrative purposes only, as it is understood that models for other types of moving platforms, and models of other types of occupant connections, and other types of occupant postures may be similarly developed.

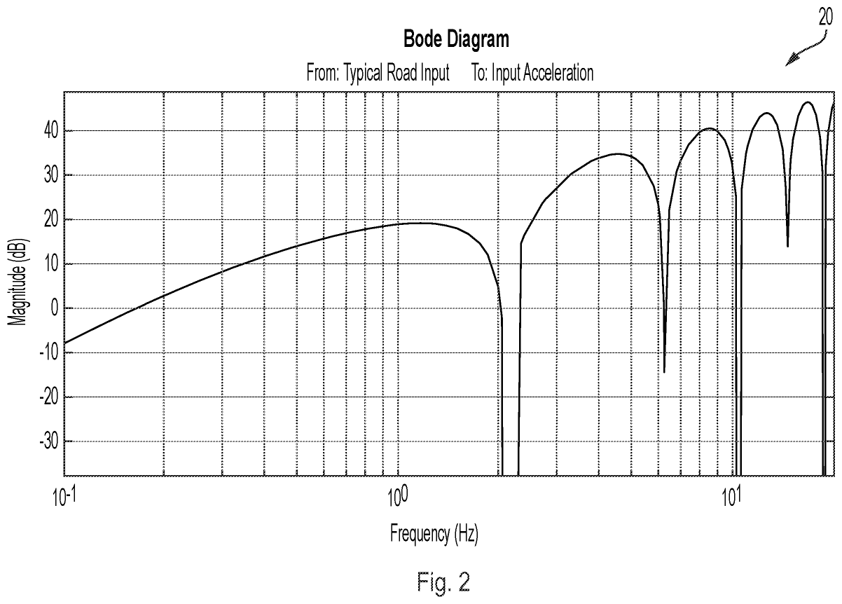

In the embodiment of a transmission chain model illustrated in FIG. 1, a road input 2 may be, for example, a hypothetical road profile following the ISO standard for typical road profile shape, an actual road profile, or a road profile devised to represent a specific or desired road type or content. The road profile may be filtered through a mechanical process 4 which may be referred to as “wheelbase filtering.” In a typical 4-wheeled road vehicle, the input from the road on each side of the vehicle may first impact the front wheel on a given side, and after a delay equal to the wheelbase divided by the forward speed of the vehicle, impact the rear wheel on the same side. This leads to a “filtering” of the input felt by the occupant in the directions that motion is experienced, for example, in the subset of motions in the heave, pitch, and roll directions. A similar principle may apply to vehicles with multiple axles such as trains, or vehicles with characteristic behavior such as ships. Similar assumptions may be made for vehicles with rigid axles where inputs on one side of the vehicle may be transmitted to the opposite side. FIG. 2 shows an exemplary modelled transfer function 20 that relates a road input and the acceleration of the base of a vehicle according to this method in a single degree of freedom.

In FIG. 1, the filtered road input may impact the vehicle and tire assembly 6, which, for example, may be simulated simply as a one-dimensional spring-mass-damper assembly with two masses, two dampers, and two springs, often referred to as a “quarter-car” model. Alternatively, a more rigorous simulation may be used that includes consideration of dynamics of additional components of the vehicle as a whole, such as a half-car model, a full-vehicle model, or a fully nonlinear multi-body model. One output of the model may be, for example, the motion of the vehicle floor that supports the base of an occupant's seat. In some embodiments, a model for this step may, for example, be a three quarter-car models in each of the three degrees of freedom most affected by road input—heave, pitch, and roll of the vehicle body.

In a further refinement step, the motion of the vehicle floor may be applied to a model of a seat system 8 shown in FIG. 1. The seat model may include, for example, a seat base, a seat cushion, and the occupant. In some embodiments, this may be modelled using a simple spring-mass system. Alternatively, a more rigorous model may be used such as, for example, a multi-body model.

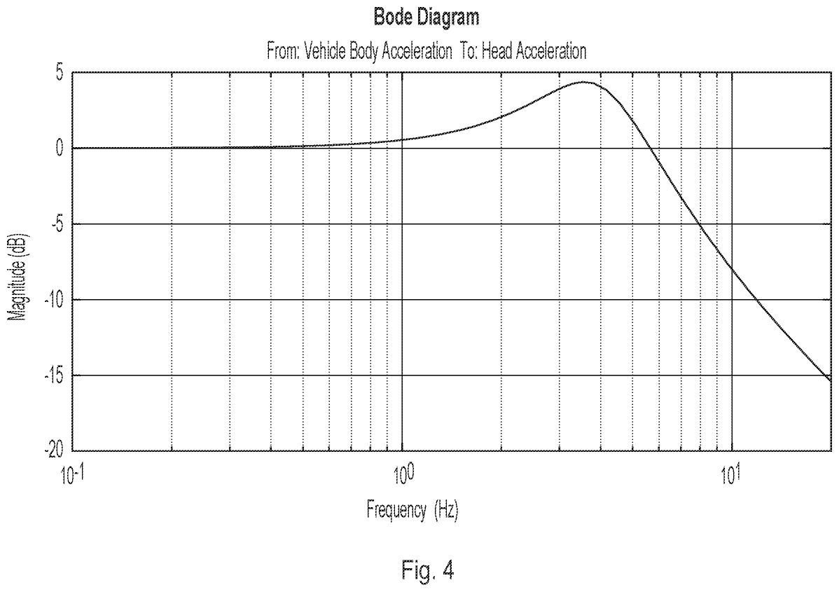

In the embodiment of FIG. 1, an occupant 10 may be sitting on the seat 8. The transfer of motion between the seat's contact surfaces and the occupant (e.g., the buttocks and the arms of the occupant) and the occupant's floor contact surfaces (e.g., the occupant's feet) to the occupant's head may be approximated by using a linear model. Alternatively, it may be approximated using a more rigorous model depending on the desired accuracy of the predicted output. FIG. 4 shows an exemplary transfer function of a seat, combined with a model of a human torso and neck, showing the expected motion of the head associated with the motion of the vehicle body or seat base.

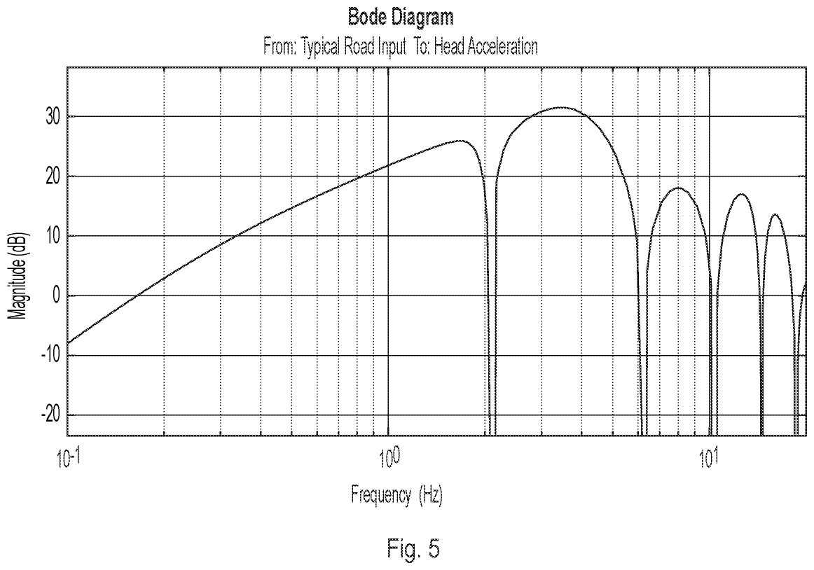

Thus, in some embodiments, the model illustrated in FIG. 1 may be used to predict occupant head motion based on road input or disturbance which may be conveyed by an intervening platform, e.g., vehicle body, vehicle floor, or a seat in the vehicle. For example, in some embodiments, the model may be a set of three single-direction models expressing the head motion expected due to road input in a road vehicle travelling over an average road, in the heave, pitch, and roll degrees of freedom. FIG. 5 shows an exemplary transfer function for a single degree of freedom model, that relates a typical road input to the expected head acceleration of an occupant.

In some embodiments, multiple performance metrics, that are relevant in reducing kinetosis, may be determined based on the expected head motion. Among these metrics may be human sensitivity to vibration 12, for example as defined by NASA during research for the space program, VOR suppression 14, discussed below, and tracking error 16, also discussed below. Performance metrics are not limited to the ones listed below, as other metrics may be selected from past experience and by combining existing metrics, as the disclosure is not limited to the metrics discussed above.

The Inventors have recognized that when a person focuses his or her gaze on a target, for example, while reading, on a word or group of letters displayed on a screen, or while looking at any particular object or a portion of an image, two mechanisms may be at play—fixation and the visual-ocular reflex (VOR).

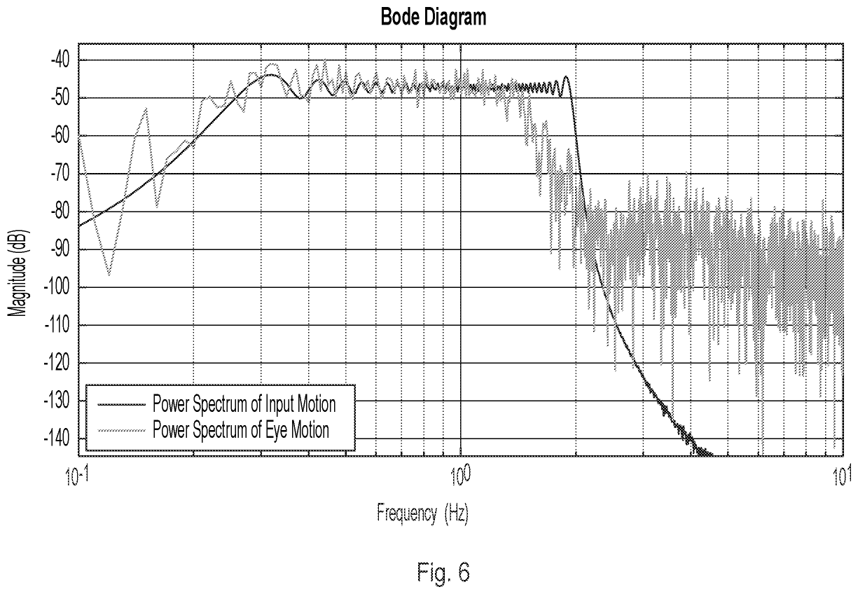

Fixation is a mechanism by which the brain attempts to move eye muscles in order to maintain focus on, for example, the desired target or series of targets when those are moving relative to a person's eye. It may function as a feedback mechanism whereby any deviation of the fixation point from the object is corrected by a voluntary or intentional corrective response. Fixation may occur when a fixation error is detected, and eye muscles are commanded to correct the error. FIG. 6 shows an exemplary power spectrum of the motion of a target a test subject is asked to fixate upon, and the corresponding motion of the test subject's gaze as measured by an eye motion sensor. FIG. 6 illustrates that up to a certain frequency, for example, in this case approximately 1.5 Hz, the eye motion follows the input motion or disturbance. Above that frequency, the eye motion may drop off and may be unable to keep up with the input motion or disturbance. This type of data may be used to model the fixation response of a typical person, or alternatively of a particular person in a particular use case and task. Fixation response typically may track a target at low frequency of motion of the target, for example below 1.5 Hz or below 1 Hz.

The visual-ocular reflex (VOR) is an involuntary mechanism by which a person's eye or eyes may respond to compensate for the head motion. The motion may be sensed by the inner ear or ears and the eye muscles may move reflexively in response to the sensed motion. This mechanism allows the brain to automatically compensate for both unintentional and intentional head motions and maintain eye-focus on a target during and despite those motions. The VOR is generally effective at compensating for motions that are above a certain threshold value or range, for example above 0.5 Hz or above 1 Hz, and may not compensate for very slow motions. The mechanism is evolutionarily suited for situations where the head is moving but the object of focus is not moving in the inertial reference frame. Such a situation may be present when a person is on a moving platform, such as for example a road vehicle, but an occupant's eyes are fixating on an object that is outside the vehicle and is not moving with the platform, e.g., the road surface or the environment, e.g., trees or building, or road signs outside of a vehicle.

The Inventors have recognized that when a person, e.g., an occupant of a vehicle, is on a moving platform and is engaged in a focal visual task, a motion of the moving platform may induce a matching or commensurate motion of the target of the task. For example, the target may be a handheld device or computer screen, book, paper, which may be directly or indirectly be attached to the platform, e.g., the vehicle body, and may follow the motion of the platform up to a frequency of above 1 Hz or above 5 Hz. In this case, the VOR may cause eye-motion that compensates for motion of the head, but in doing so may move the gaze, or focus, off the intended target since, as discussed above, the target may be moving as well.

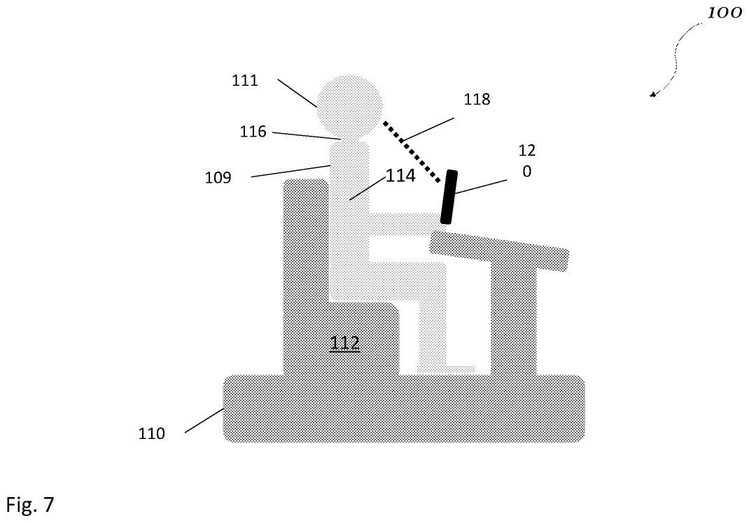

FIG. 7 illustrates a work station 100 in a moving vehicle. Occupant 109 is seated on seat 112 which may be attached to a platform 110 that is fixedly attached to the vehicle body (not shown). The occupant may be sitting on a seat attached to the platform, standing on the platform, or otherwise supported by the platform. The motion of the occupant's head 111 may be the result of a combination of the motion induced by the motion of the platform 110, as modified and/or mitigated by various interposed components or portions of an occupants body (e.g., a seat 112, and/or the occupant's torso 114 and neck 116), and any other unintentional or intentional motion of the neck and head. The occupant's gaze 118 may be fixated on a target 120 that may be handheld or attached, directly or indirectly, to the platform 110. However, the motion of target 120 may be, at least in part, also induced by the motion of the platform 110. The VOR in this use may induce a counterproductive involuntary eye motion which may cause the gaze to wander off the target since the target is also moving. This situation may require a fixation effort by the eye muscles, to at least partially cancel the effect of VOR, and to maintain on-target fixation of the gaze.

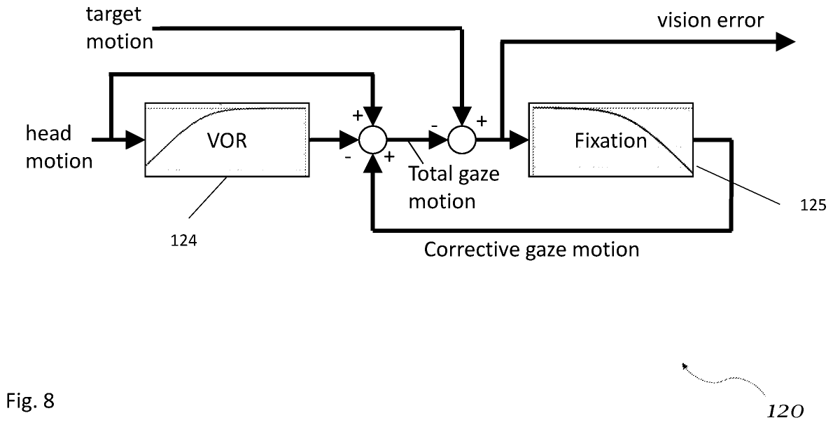

The block diagram in FIG. 8 is an illustration of the effect of VOR during fixation on a target in a moving vehicle. Head motion may cause a VOR response 124. In the example illustrated in FIG. 8, the total gaze motion in the absolute reference frame is the sum of the head motion and any corrective motion (due to tracking) of the eye muscles, minus the involuntary corrective effect of the VOR. This gaze motion may subtract from the actual target motion to create a vision error, which may be the difference between where the occupant is intending to look and where their gaze actually points. The fixation 125 originated by the brain may attempt to minimize this error by creating a corrective gaze motion in a feedback loop.

The Inventors have recognized multiple causes of kinetosis that may be addressable through shaping or controlling of the platform motion. In one embodiment, a cause may be the effort associated with compensating for the VOR.

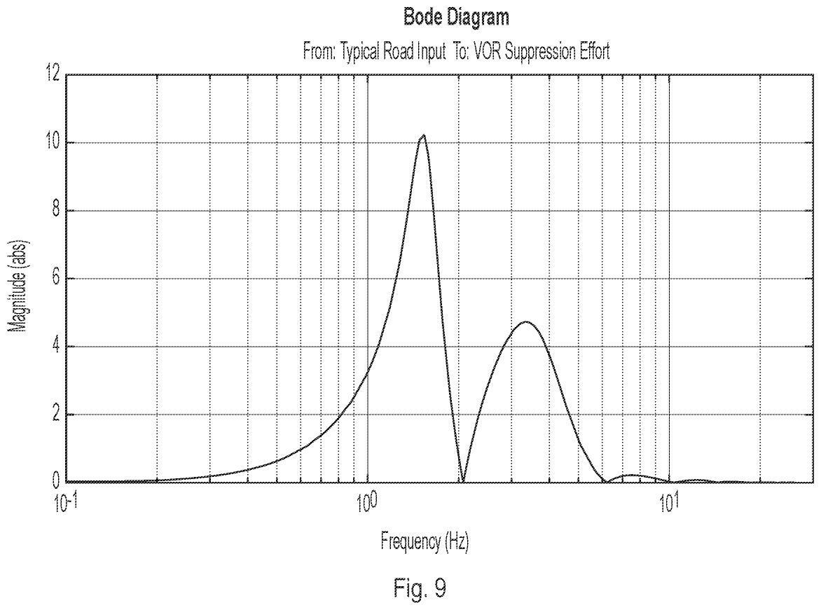

Using the model described above, as well as a model of the VOR and of the fixation response, a complete model may be developed relating motion of the platform to the effort involved in correcting for the VOR response (the corrective gaze motion). FIG. 9 shows an example of the response of such a model, highlighting the importance of some frequencies over others.

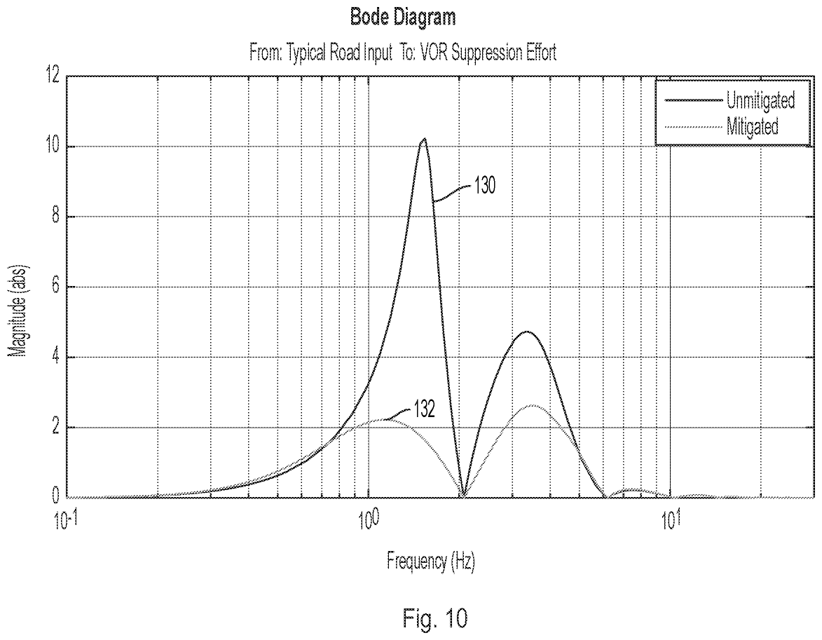

In some embodiments of a control system, a transfer function such as the transfer function illustrated in FIG. 9, may be used as a shaping function to determine the frequencies and amplitudes that require attention. It may for example be used as a cost function shaping filter in the control design step, or as an input filter applied to the input signal when designing the optimal control system for platform 110 in FIG. 7. A model of the control system may be used to predict the performance of the controlled platform, resulting for example in the response such as, for example, shown in FIG. 10. In FIG. 10, the “unmitigated” response 130 and the “mitigated” response 132 of the platform, with controls tuned to minimize the VOR suppression effort, are compared.

In some embodiments, a cause of kinetosis or occupant discomfort may be due to vision error illustrated in FIG. 8. This vision error may not only lead to kinetosis, as a person's brain tries perform a fixation task appropriately in a moving vehicle, but may also lead to a reduction in overall productivity that may affect the task performance of an occupant. For example, a continued vision error may affect the occupant's ability to read, understand, or write while on the moving platform, which may be an impediment when travelling in a vehicle, for example, in an autonomous vehicle.

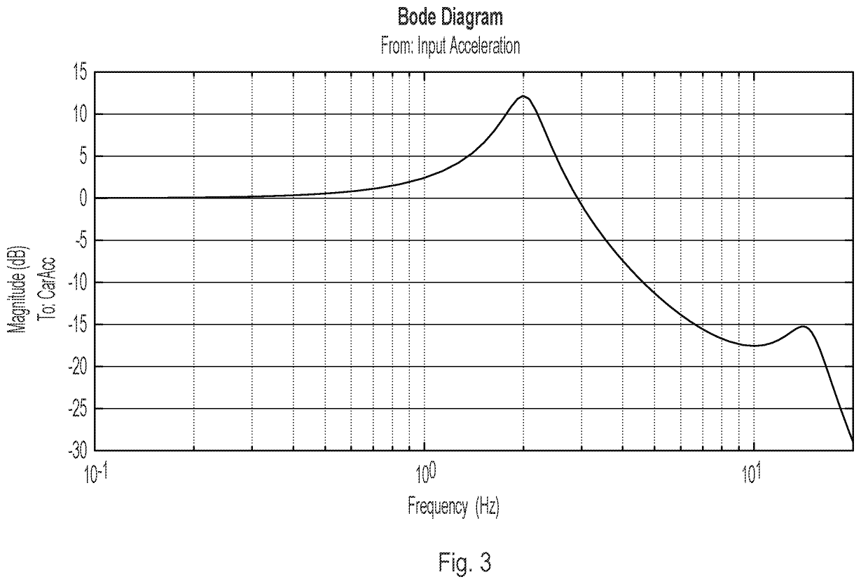

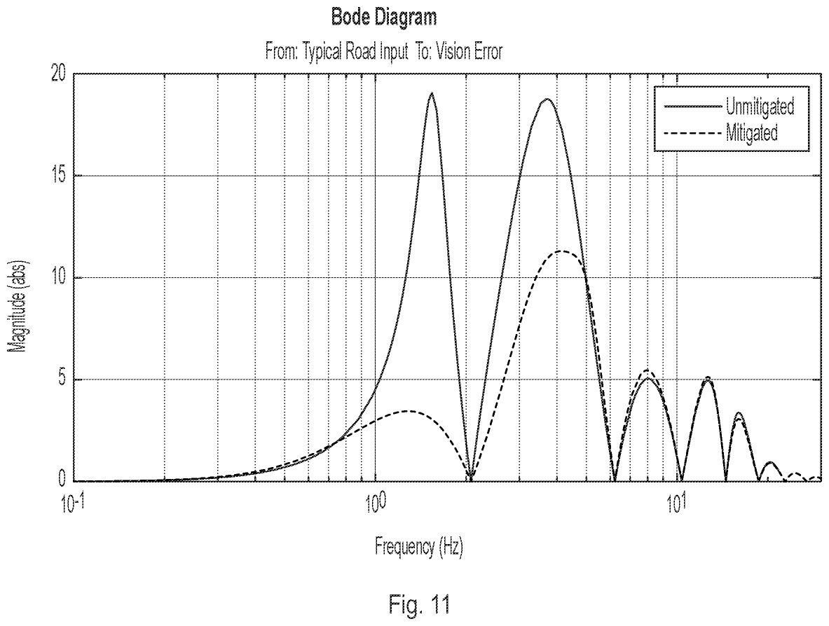

The response of the system, to a typical input and a resulting vision error, may be estimated by using the models described in FIGS. 1-8. FIG. 11 shows an exemplary complete modelled transfer function from a typical input to a vision error in one particular degree of freedom, and illustrates the improvement that may result from a strategy focused on reducing vision error.

A VOR effort and a vision error may be sensed using an eye tracking device and either calculating or measuring the motion of the gaze with respect to a target. This may be done in a test environment to understand the use case and shape the response and the control system, or it may be done in real time and used as a feedback sensor, for example by using a non-contact and non-intrusive sensor such as a camera pointing at the occupant such as a front-facing camera on a mobile device.

In some embodiments, a cause of kinetosis and discomfort may be head motion. The human balance organs in the inner ear are sensitive to even small motions, and from an evolutionary standpoint did not evolve in a setting where humans used moving platforms, to transport themselves from one place to another, where the target moves simultaneously with the occupant's head. This evolutionary history may lead to a sensitivity to head motion, especially when that motion is imparted by external and unpredictable sources such as a moving platform when the occupant is not paying attention to the environment.

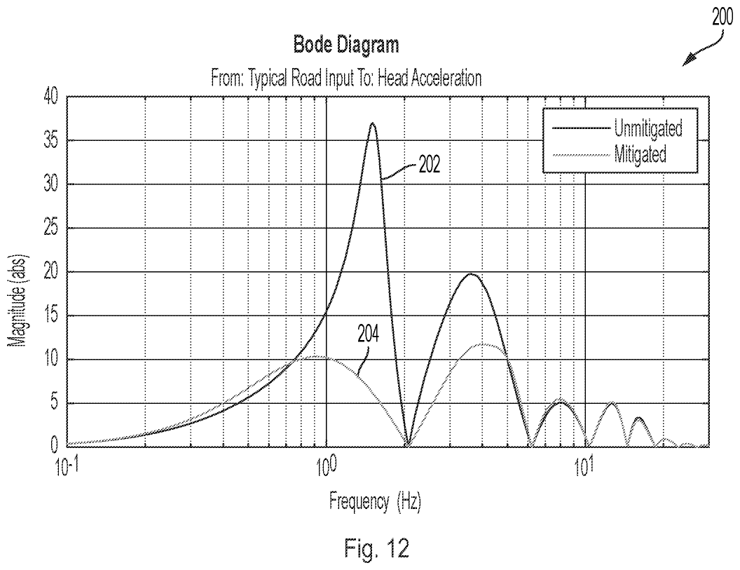

In some embodiments, the characteristics of the motion of a vehicle occupant's head may be determined by using the model illustrated in FIG. 1, based on the motion imparted to the vehicle and/or the platform supporting the occupant, e.g., as illustrated in FIG. 7. FIG. 12 illustrates an exemplary transfer function 200 of a model with a single degree of freedom predicting head acceleration as a function of typical road input for an exemplary road vehicle.

It shows a comparison of a typical vehicle's behavior 202 (labelled “unmitigated”) to a controlled vehicle 204 (labelled “mitigated”).

A head motion transfer function may be determined by using a simple or a rigorous simulation (e.g., computer simulations), but may also be determined empirically using motion sensors (e.g., contact sensors, such as accelerometers, or non-contact sensors, such as vision sensors, for example the front-facing camera on a mobile device). In some embodiments, sensor measurements may be used as a feedback signal to instantaneously correct or compensate for any motion that may aggravate kinetosis, or as a performance sensor to adapt the tuning of the control system.

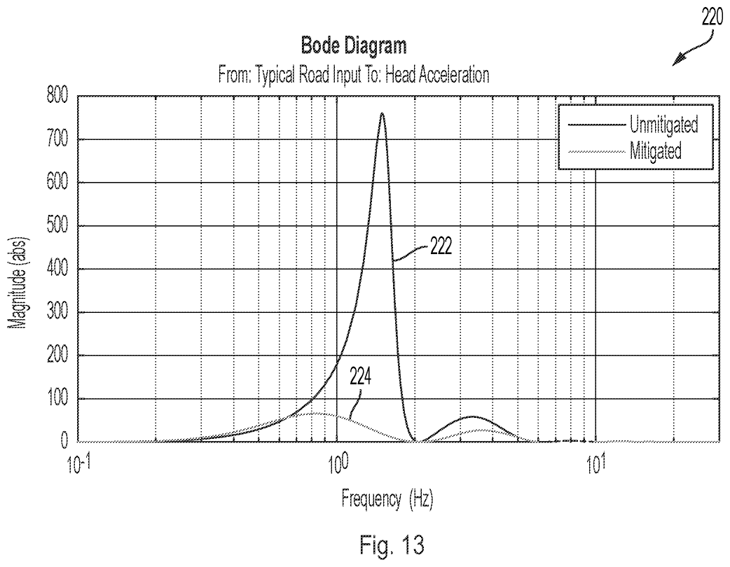

In some embodiments, kinetosis may result from or be aggravated by a combination of rotational and linear motions of the head. Such combinations may be predicted using a model that captures multiple degrees of freedom of motion, or may be measured and used either as a direct feedback signal or as an indirect signal used for adaptive controls strategies. In certain embodiments, undesirable frequencies for combined motions may be predicted, for example, by multiplying a modelled response function (e.g., for a typical road input to head motion in one direction, for example heave), with a modelled response function from typical road input to head motion in a different direction, for example roll. FIG. 13 shows a sample transfer or response function 220 determined in this way for an unmitigated typical vehicle model 222, and for a mitigated response 224 using a control system tuned to suppress combined head motions.

Combined head motions may be predicted as described, using a model of the system, but may also be sensed during operation and used in a feedback control loop with the platform control system.

In some embodiments, kinetosis may result from the inability of a person's brain to predict motions that are sensed by the inner ear. The prediction method may be described as the brain building a reference model of the motion and comparing the expected motion with the sensed motion. This mechanism may explain certain aspects of kinetosis. For example, it may explain why focusing on a distant point in a moving vehicle or ship may help delay the onset or reduce the severity of kinetosis, and it also explains why on certain types of platforms, for example large ships, people have been able to adapt and not feel motion sick after a while. This may be because a motion of a ship is largely predictable, since it is heavily resonant and acts like a resonant low-pass filter on the random wave input. For example, a motion of a ship when encountering a wave will have strong content at a primary frequency that can be derived from the ship's length and other characteristics, and every upward motion is invariably followed by a predictable downward motion and successively smaller following upward motion in turn. In some embodiments, this principle may be applied to road vehicles and trains. For example, if a vehicle does not have a highly resonant behavior since it amplifies input motions that way; a road vehicle with low damping characteristics may be strongly susceptible to road input near the primary resonance, which may often lead to discomfort. The inventors have recognized that this may in turn be beneficial for the predictability and/or modeling of the motion, and that it may lead to a reduction in kinetosis. In some embodiments, therefore, a control strategy may include causing a highly resonant behavior of the vehicle in any given direction of motion for all small motions, and a progressively less resonant behavior for larger motions in order to reduce incidences of peak motion amplitude and/or acceleration. This may be achievable, for example, with a variable damping strategy that uses low damping values near ride height and at low damper velocities, and that progressively increases the damping value at larger amplitude and larger damper velocity. This may, for example, be achieved by using an algorithm for actuator travel management, or by using mechanical devices such as elastomeric bump stops or rebound springs.

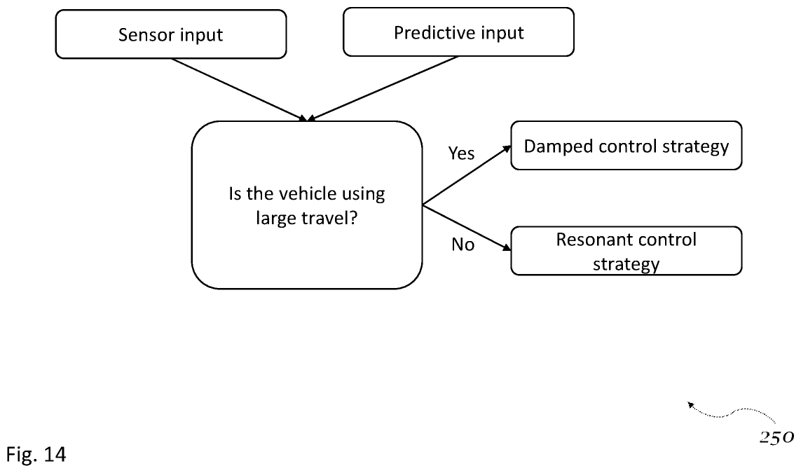

In some embodiments, this concept may be implemented by using an active suspension system that is configured to modify the behavior of the platform or vehicle. In such embodiments, a control strategy may be imposed that increases the resonant behavior of the vehicle and creates an artificially low primary body resonance. In some embodiments, vehicles may have a primary resonance tuned to be between 0.8 Hz and 2.5 Hz, or tuned to be in the range of 1.0 Hz-1.2 Hz. A lower body resonance provides a more perceivable resonant feel but leads to larger travel excursions for a given road input. In some embodiments, a control strategy as illustrated in FIG. 14 may be used to mitigate either anticipated or sensed travel excursions that exceed a set threshold, or a variable threshold, and modify the control strategy accordingly to maximize the resonant feel of the vehicle but without incurring large travel excursions. It should be noted that in some embodiments, the switch between the resonant and damped control strategy may be discrete or may be on a sliding scale, for example, by using a variable gain setpoint according to the amount of travel used.

In some embodiments, kinetosis may be caused by the unpredictable nature of motions a vehicle occupant is exposed to in a moving platform or vehicle. For example, for an occupant in a road vehicle who is not observing the road ahead of the vehicle, the motion experienced in the vehicle may appear largely randomized and unpredictable. For example, in such a scenario, a vehicle occupant may be unable to anticipate turning motions, accelerations (positive or negative), and large road inputs. In some embodiments, providing cues may allow a vehicle occupant to anticipate motions they are exposed to and thus delay the onset or reduce the severity of kinetosis if and when it occurs. In some embodiments a cueing strategy may include exposing a vehicle occupant to a reduced or mitigated version of the upcoming motion. In such a strategy, a left turn may be anticipated through a slight roll motion of the vehicle to the right, since left turns lead to a vehicle roll motion to the right. Alternatively, the cue may include a slight roll motion to the left, since left turns involve acceleration pulling the occupant to the left. An upcoming large road input may be anticipated through a reduced version of the expected motion. For example, a large road event may be anticipated through a reduced version of only the characteristic motions that may induce kinetosis. As an example, before crossing a speedbump in a road vehicle, the control system may impart a slight upward motion of the vehicle, followed by a slight drop back to its normal ride height. This sequence of motions may cause the occupant to subconsciously anticipate the larger motion, and thus reduce their susceptibility to kinetosis.

The mitigation strategies explained here may be implemented in multiple ways. In some embodiments, the platform control system may be designed using a model of the system, and optimizing the selected performance metric in the model, thus providing an optimized performance for the system in so far as it behaves like the model. In some embodiments, this model may be as simple as one or multiple single degree of freedom models, such as a set of quarter-car models for a road vehicle.

The platform control system may be a control strategy that applies a control signal in response to a feedback sensor, or it may be a control strategy that is implemented using a series of design choices on a set of physical components. For example, a model may be built to predict the relationship between a typical road input and a selected performance metric, and then system parameters may be optimized to produce the best expected performance within a range of possible choices for physical components such as springs and dampers.

The mitigation strategies may also be applied by using sensors measuring the performance metric, such as for example sensors measuring the head motion, the eye motion, or vision error.

A sensor may be used in a feedback loop to improve performance in conjunction with an active suspension system of the platform. For example, a vehicle with an active suspension system or a semi-active suspension system may use a signal from a front-facing camera on a handheld device, or from an internal camera installed inside the vehicle, to estimate the head motions, combined head motions, vision errors, and/or VOR suppression effort. In some embodiments, it may be used during tuning to pre-determine the optimal performance, for example, to select desired calibrations for the tuning parameters. This strategy may be implemented, for example, if the sensor used to estimate the performance metric is expensive, intrusive, or not always available. In some embodiments, it may be used in an adaptive way, for example, to modify parameter settings when a large amount of motion is detected, or when the sensor indicates a significant change in the performance metric, or when the sensor or a user input shows that the occupant is performing a focal visual task. In some embodiments, it may be used as a direct feedback sensor to control system motion, for example to minimize vehicle roll motion when large occupant head motions are sensed.

The mitigation strategies may also be applied in a predictive or pre-emptive way, by analyzing upcoming input and adapting the control strategy to optimize performance for an anticipated road input or disturbance. For example, a road vehicle with predictive information on the upcoming road, for example using a camera, radar, lidar system, or using information from previous drives or other vehicles (e.g., crowd sourced road data), may analyze the upcoming road information and adapt the control strategy to maximize performance in at least one selected metric while also satisfying other system limits, e.g., reducing power consumption.

In some embodiments, the onset of kinetosis may be detected, for example, by using a sensor such as a camera to detect a change in skin pallor or flushing, and/or sensors to measure heart rate, breathing, or other metrics that may correlate with kinetosis. In some embodiments, an improvement in kinetosis may be achieved at the expense of a reduction in other metrics, that may be relevant to the occupants at other times when kinetosis is not a concern, for example, overall comfort, handling, vehicle component wear etc. In some embodiments, a system may be used to detect the onset of kinetosis, and a parameter setting may be chosen to favor mitigation over e.g., handling for at least some subsequent period of time, which may be predetermined or preset. In some embodiments, a strategy may be used in a semi-autonomous vehicle, when the occupant is driving and is focusing on the road ahead, as sensed for example by an inward-facing camera, the parameter settings may be chosen to favor, for example, road holding and road feel. Alternatively, when the occupant is not driving and is instead focusing on, e.g., a handheld or vehicle-mounted target, the parameter settings may be modified to favor kinetosis reduction and comfort. In some embodiments, this setting may also be modified by a user or the target operator in a situation where multiple occupants may be inside the same vehicle. In some embodiments, parameters settings that favor kinetosis reduction may not be implemented if it is determined that the vehicle occupants are not sensitive to kinetosis at all or in view of anticipated road induced disturbances. This determination may be based on, for example, previously acquired information about particular passengers and/or information collected from the passengers by using a user interface.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

1. A method of operating a vehicle, the method comprising:
(a) transporting an occupant of the vehicle over a road surface, wherein the occupant is seated on a seat in the vehicle;
(b) with a first sensor, measuring a road-surface-induced disturbance of a portion of the vehicle;
(c) based on the measurements in step (b), determining an aspect of a motion of the occupant's head with a first model that relates the motion of the occupant's head to the road-surface-induced disturbance in step (b);
(d) based on the determination in step (c), controlling a motion of the seat with a microprocessor-based controller; and
(e) controlling the aspect of the motion determined in (c) with the motion in step (d).

2. The method of claim 1, wherein the aspect of a motion of the occupant's head is selected from the group consisting of pitch motion of the occupant's head, roll motion of the occupant's head, and heave motion of the occupant's head.

3. The method as in claim 1, wherein the controlling of the aspect in step (e) includes controlling an amplitude of the motion.

4. The method of claim 3, wherein the amplitude is controlled in a frequency range between 1 Hz and 9 Hz.

5. The method of claim 3, wherein the amplitude is controlled in a frequency range between 3 Hz and 8 Hz.

6. The method as in claim 1, wherein a seat actuator, interposed between the seat and a vehicle floor, is used to control the motion of the seat in step (d).

7. The method as in claim 1, wherein a vehicle active suspension actuator interposed between an unsprung mass of the vehicle and a sprung mass of the vehicle, is used to control the motion of the seat in step (d).

8. The method as in claim 1, wherein the first model includes a first transfer function that relates a movement of the occupant's head to a movement of the seat.

9. The method of claim 8, wherein the first model includes a second transfer function that relates the movement of the seat to a movement of the sprung mass.

10. The method of claim 9, wherein the first model includes a third transfer that relates the movement of the sprung mass to a movement of the unsprung mass.

11. The method as in claim 1, wherein the microprocessor-based controller in (d) includes feedback controller that receives information from the first model.

12. A method of operating an active suspension system of a vehicle, the method comprising:
receiving a signal indicative of a motion of an occupant of the vehicle;
operating at least one actuator of the active suspension system at least partially based on the signal.

13. The method of claim 12, wherein the signal is at least partially based on the output of a sensor.

14. The method of claim 12, wherein the signal is at least partially based on the output of a model.

15. The method of claim 12, wherein the motion of the occupant is a motion of the occupant's head.

16. The method of claim 12, wherein the motion of the occupant is a motion of the occupant's eye.

17. The method of claim 12, wherein the motion of the occupant is at least partially based on predictive information about an upcoming road.

18. (canceled)

19. A method of operating a vehicle, the method comprising:
(a) transporting an occupant of the vehicle over a road surface, wherein the occupant is seated on a seat in the vehicle and performing a focal visual task;
(b) with a first sensor, measuring a road-surface-induced disturbance of a portion of the vehicle;
(c) based on the measurements in step (b), determining, with a first model, a value of a parameter associated with a motion of an eye of the occupant during performance of the focal visual task wherein the first model relates the motion of the occupant's eye to the disturbance in step (b);
(d) based on the determination in step (c), controlling a motion of the seat with a microprocessor-based controller; and
(e) controlling the value of the parameter determined in (c) with the motion in step (d).

20. The method of claim 19, wherein the parameter in step (c) is selected from the group consisting of fixation rate, a forward saccade ratio, a saccade amplitude, a backward saccade amplitude, vision error, and VOR suppression.

21. The method of claim 19, wherein a seat actuator interposed between the seat and a vehicle floor is used to control the motion of the seat in step (d).

22. The method of claim 19, wherein a vehicle active suspension actuator interposed between an unsprung mass of the vehicle and a sprung mass of the vehicle is used to control the motion of the seat in step (d).

23. The method of claim 19, wherein first model includes a first transfer function that relates a movement of the occupant's eye to the motion of the occupant's head.

24. The method of claim 19, wherein first model includes a second transfer function that relates a movement of the occupant's head to a movement of the seat.

25-29. (canceled)
